SEQUENCE LISTING (8 sequences)

SEQ ID NO: 1 - protein, 274 aa, Humicola lanuginosa
  VARIANT (1)..(274)
  mat_peptide (6)..(274)
SPIRREVSQDLFNQFNLFAQYSAAAYCGKNNDAPAGTNITCTGNACPEVEKADATFLYSFEDSGVGDVTGFLALDNTNKLIVLSFRGSRSIENWIGNLNFDLKEINDICSGCRGHDGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVPVRRRDIVKIEGIDATGGNNQPNIPDIPAHLWYFGLIGTCL

SEQ ID NO: 2 - protein, 269 aa, Humicola lanuginosa [sequence omitted]

SEQ ID NO: 3 - protein, 274 aa, Bacillus licheniformis
  mat_peptide (1)..(274)
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

SEQ ID NO: 4 - protein, 188 aa, Nocardiopsis sp.
  mat_peptide (1)..(188)
ADIIGGLAYTMGGRCSVGFAATNAAGQPGFVTAGHCGRVGTQVTIGNGRGVFEQSVFPGNDAAFVRGTSNFTLTNLVSRYNTGGYATVAGHNQAPIGSSVCRSGSTTGWHCGTIQARGQSVSYPEGTVTNMTRTTVCAEPGDSGGSYISGTQAQGVTSGGSGNCRTGGTTFYQEVTPMVNSWGVRLRT

SEQ ID NO: 5 - protein, 188 aa, Nocardiopsis dassonvillei subsp. dassonvillei
  mat_peptide (1)..(188)
ADIIGGLAYYMGGRCSVGFAATNSAGQPGFVTAGHCGTVGTGVTIGNGTGTFQNSVFPGNDAAFVRGTSNFTLTNLVSRYNSGGYQSVTGTSQAPAGSAVCRSGSTTGWHCGTIQARNQTVRYPQGTVYSLTRTNVCAEPGDSGGSFISGSQAQGVTSGGSGNCSVGGTTYYQEVTPMINSWGVRIRT

SEQ ID NO: 6 - protein, 513 aa, Bacillus stearothermophilus
  VARIANT (1)..(513)
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGKAWDWEVDTEFGNYDYLMYADLDMDHPEVVTELKNWGKWYVNTTNIDGFRLDAVKHIKFSFFPDWLSYVRSQTGKPLFTVGEYWSYDINKLHNYITKTDGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 7 - protein, 481 aa, Bacillus licheniformis
  VARIANT (1)..(481)
VNGTLMQYFEWYTPNDGQHWKRLQNDAEHLSDIGITAVWIPPAYKGTSQADVGYGAYDLYDLGEFHQKGTVRTKYGTKGELQSAIKSLHSRDINVYGDVVINHKGGADATEDVTAVEVDPADRNRVISGEHLIKAWTHFHFPGRGSTYSDFKWYWYHFDGTDWDESRKLNRIYKFQGKTWDWEVSNEFGNYDYLMYADIDYDHPDVVAEIKRWGTWYANELQLDGFRLDAVKHIKFSFLRDWVNHVREKTGKEMFTVAEYWSNDLGALENYLNKTNFNHSVFDVPLHYQFHAASTQGGGYDMRKLLNGTVVSKHPLKSVTFVDNHDTQPGQSLESTVQTWFKPLAYAFILTRESGYPQVFYGDMYGTKGDSQREIPALKHKIEPILKARKQYAYGAQHDYFDHHDIVGWTREGDSSVANSGLAALITDGPGGAKRMYVGRQNAGETWHDITGNRSEPVVINSEGWGEFHVNGGSVSIYVQR

SEQ ID NO: 8 - protein, 483 aa, Bacillus sp.
  VARIANT (1)..(483)
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVKAVEVNPNNRNQEVSGEYTIEAWTKFDFPGRGNTHSNFKWRWYHFDGVDWDQSRKLNNRIYKFRGKGWDWEVDTEFGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQKHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNKAGTVTINADGWGNFSVNGGSVSIWVNK

(19) United States
(12) Patent Application Publication
(10) Pub. No.: US 2009/0047266 A1
(43) Pub. Date: Feb. 19, 2009

(54) Lipases for Pharmaceutical Use

(75) Inventors: Allan Svendsen, Horsholm (DK); Peter Colin Gregory, Hannover (DE); Kim Borch, Birkerod (DK)

Correspondence Address: NOVOZYMES NORTH AMERICA, INC., 500 FIFTH AVENUE, SUITE 1600, NEW YORK, NY, 10110, US

(73) Assignees: Novozymes A/S, Bagsvaerd (DK); Solvay Pharmaceuticals Hans-Boeckler, Hannover (DE)

(21) Appl. No.: 11/917,584

(22) PCT Filed: Jun. 16, 2006
(86) PCT No.: PCT/DK2006/000352

§ 371 Date: Jan. 23, 2008

Related U.S. Application Data
(60) Provisional application No. 60/694,173, filed on Jun. 27, 2005.

(30) Foreign Application Priority Data
Jun. 24, 2005 (DK) PA 2005 00929

(51) Int. Cl. A61K 38/54; A61K 38/46; A61P 3/10; A61P 11/00; A61P 1/00

(52) U.S. Cl. 424/94.2; 424/94.6

(57) ABSTRACT

The pharmaceutical use of lipases related to a variant of the Thermomyces lanuginosus (Humicola lanuginosa) lipase comprising amino acids 1-269 of SEQ ID NO: 1, optionally in combination with a protease and/or an amylase. Examples of medical indications are: Treatment of digestive disorders, pancreatic exocrine insufficiency (PEI), pancreatitis, cystic fibrosis, diabetes type I, and/or diabetes type II. The lipases of the invention have an improved efficacy in vivo, are stable against protease-degradation, and/or are stable in the presence of bile salts.

### TECHNICAL FIELD

The present invention relates to the pharmaceutical use of lipases related to a Thermomyces lanuginosus (synonym: Humicola lanuginosa) lipase variant comprising amino acids 1-269 of SEQ ID NO: 1. The lipases may be used in combination with a protease and/or an amylase. Examples of medical indications are: Treatment of digestive disorders, pancreatic exocrine insufficiency (PEI), pancreatitis, cystic fibrosis, diabetes type I, and/or diabetes type II.

### BACKGROUND ART

Several commercial medicaments in the form of pancreatic enzyme supplements are known for the treatment of pancreatic exocrine insufficiency. The active ingredients of these products are digestive enzymes, mainly amylase, lipase and protease, which are normally produced in the pancreas and excreted to the upper part of the small intestine (the duodenum). The enzymes used in such medicaments mainly derive from bovine or swine pancreas, however there are also products on the market with microbial enzymes, e.g. the product Nortase® which contains a lipase from Rhizopus oryzae, a protease from Aspergillus oryzae, and an amylase from Aspergillus oryzaie.

U.S. Pat. No. 5,614,189 (EP 600868) describes the use of, i.a., a lipase derived from Humicola lanuginosa in pancreatic enzyme replacement therapy, for example in the treatment of patients suffering from cystic fibrosis. This lipase is from Humicola lanuginosa DSM 4109 and has the amino acid sequence of amino acids 1-269 of SEQ ID NO: 2.

WO 00/54799 describes the use of physiologically acceptable enzyme mixtures having lipolytic, proteolytic and amylolytic activity in the treatment of diabetes mellitus type I and II.

WO 02/060474 describes the use of a concentrated lipase from Rhizopus delemar, a neutral protease from Aspergillus melleus, and an amylase from Aspergillus oryzae in the treatment of maldigestion.

WO 01/62280 describes the use of a non-fungal lipase crystal crosslinked with a multifunctional crosslinking agent, a protease, and an amylase, wherein the lipase crystal is active at a pH range from about 2.0 to 9.0, for treating or preventing a gastrointestinal disorder in a mammal. A preferred lipase is from Pseudomonas, preferred amylases are from Bacillus or Aspergillus, preferred proteases are bromelain, papain or ficin.

EP 0828509 describes the use of certain acid-stable amylases, optionally in combination with certain acid-stable lipases and/or proteases, in the treatment of exocrine pancreas insufficiency. A preferred amylase is from Aspergillus niger, and preferred lipases are from Rhizopus arrhizus or Rhizopus javanicus.

WO 00/60063 describes a number of variants of the Humicola lanuginosa lipase and their use in detergents. The lipase having amino acids 1-269 of SEQ ID NO: 1 herein is specifically described, however not its pharmaceutical use.

WO 04/111216 and EP 1428874 both disclose variants of SEQ ID NO: 2, including variants of SEQ ID NO: 1, but not the pharmaceutical use thereof.

There is a need in the art for alternative, preferably improved, enzymes for pharmaceutical use.

### SUMMARY OF THE INVENTION

The present invention provides alternative, preferably improved, enzymes for pharmaceutical use, viz. new lipases, amylases, and proteases. Preferably, the enzymes for use according to the invention have an improved efficacy in vivo and/or in vitro; an improved pH-stability profile; an improved pH-activity profile; are stable against degradation by proteases; are stable in the presence of bile salts; and/or have a reduced allergenicity.

The present invention relates to a lipase for use as a medicament, wherein the lipase has at least 90% identity to amino acids 1-269 of SEQ ID NO: 1, with the proviso that the lipase is not amino acids 1-269 of SEQ ID NO: 2. The lipase may be used in combination with a protease, and/or an amylase.

The invention also relates to the use of such lipases for the manufacture of a medicament for the treatment of digestive disorders, PEI, pancreatitis, cystic fibrosis, diabetes type I, and/or diabetes type II, these uses optionally further comprising the use of a protease, and/or an amylase.

The invention furthermore relates to a pharmaceutical composition comprising such lipases, together with at least one pharmaceutically acceptable auxiliary material, optionally including a protease and/or an amylase.

The invention also relates to a method for the treatment of digestive disorders, PEI, pancreatitis (acute and/or chronic), cystic fibrosis, diabetes type I, and/or diabetes type II, by administering a therapeutically effective amount of such lipases, optionally together with a protease and/or an amylase.

### DETAILED DESCRIPTION OF THE INVENTION

### Enzymes

The present invention relates to the pharmaceutical use of a lipase, wherein the lipase has, or comprises, an amino acid sequence which has at least 90% identity to amino acids 1-269 of SEQ ID NO: 1, with the proviso that the lipase is not amino acids 1-269 of SEQ ID NO: 2.

In a particular embodiment, a) the lipase comprises amino acids 1-269 of SEQ ID NO: 1, or b) the lipase is a variant of amino acids 1-269 of SEQ ID NO: 1, wherein the variant differs from amino acids 1-269 of SEQ ID NO: 1 by no more than twenty-five amino acids, and wherein: (i) the variant comprises at least one conservative substitution and/or insertion of one or more amino acids as compared to amino acids 1-269 of SEQ ID NO: 1; and/or (ii) the variant comprises at least one small deletion as compared to amino acids 1-269 of SEQ ID NO: 1; and/or (iii) the variant comprises at least one small N- or C-terminal extension as compared to amino acids 1-269 of SEQ ID NO: 1; and/or (iv) the variant is an allelic variant of the lipase having amino acids 1-269 of SEQ ID NO: 2; and/or (v) the variant is a fragment of the lipase having amino acids 1-269 of SEQ ID NO: 1.

The invention also relates to the use of such lipases for the manufacture of a medicament for the treatment of digestive disorders, PEI, pancreatitis (acute and/or chronic), cystic fibrosis, diabetes type I, and/or diabetes type II. The invention furthermore relates to a pharmaceutical composition comprising such lipases, together with at least one pharmaceutically acceptable auxiliary material, as well as to a method for the treatment of the above-mentioned diseases, by administering a therapeutically effective amount of such lipases. The lipase comprising amino acids 1-269 of SEQ ID NO: 1 is itself a variant of the lipase of Humicola lanuginosa (Thermomyces lanuginosus) DSM 4109 (SEQ ID NO: 2).

In what follows, the lipase for use in the compositions, methods and uses of the invention is referred to as the “lipase of the invention.”

In the present context, a lipase means a carboxylic ester hydrolase EC 3.1.1.-, which includes activities such as EC 3.1.1.3 triacylglycerol lipase, EC 3.1.1.4 phospholipase A1, EC 3.1.1.5 lysophospholipase, EC 3.1.1.26 galactolipase, EC 3.1.1.32 phospholipase A1, EC 3.1.1.73 feruloyl esterase. In a particular embodiment, the lipase is an EC 3.1.1.3 triacylglycerol lipase. The EC number refers to Enzyme Nomenclature 1992 from NC-IUBMB, Academic Press, San Diego, Calif., including supplements 1-5 published in Eur. J. Biochem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively. The nomenclature is regularly supplemented and updated; see e.g. the World Wide Web at http://www.chem.qmw.ac.uk/iubmb/enzyme/index.html.

The lipase of the invention as defined above does not encompass the lipase having amino acids 1-269 of SEQ ID NO: 2. The latter sequence differs from amino acids 1-269 of SEQ ID NO: 1 by the double-substitution R231T+R233N. The expression “the double substitution R231T+R233N” in SEQ ID NO: 1 refers to a variant of SEQ ID NO: 1 in which the two arginine residues (Arg, or R) in positions 231 and 233, respectively, have been replaced or substituted by threonine (Thr, or T) and asparagine (Asn, or N), respectively. The term “position” refers to the positive amino acid residue numbers in SEQ ID NO: 1 of the sequence listing. These two substitutions are not conservative, as defined below (since they replace two basic amino acids with two polar amino acids).

Accordingly, in a particular embodiment, the lipase of the invention does not have the amino acid sequence consisting of amino acids 1-269 of SEQ ID NO: 2, which sequence corresponds to SEQ ID NO: 1 in which the double-substitution R231T+R233N has been made.

Lipases comprising conservative substitutions, insertions, deletions, N-terminal extensions, and/or C-terminal extensions, as well as lipase fragments as compared to the sequence of amino acids 1-269 of SEQ ID NO: 1 can be prepared from this molecule by any method known in the art, such as site-directed mutagenesis, random mutagenesis, consensus derivation processes (EP 897985), and gene shuffling (WO 95/22625, WO 96/00343), etc. Such lipases may also be hybrids, or chimeric enzymes.

The variant lipase of the invention of course has lipase activity. In a particular embodiment, the specific activity of the variant lipase is at least 50% of the specific activity of the lipase having amino acids 1-269 of SEQ ID NO: 1. In additional particular embodiments, the specific activity of the variant lipase is at least 60, 70, 75, 80, 85, 90, or at least 95% of the specific activity of the lipase having amino acids 1-269 of SEQ ID NO: 1. The specific activity may be measured using any of the lipase assays of Example 1 herein, but is preferably measured in LU/mg enzyme protein using the LU-assay of Example 1, and determining enzyme protein content by amino acid analysis as described in Example 5.

The amino acid changes allowed for the lipase variant of the invention are of a minor nature, that is conservative amino acid substitutions or insertions that do not significantly affect the folding and/or activity of the protein, preferably a small number of such substitutions or insertions; small deletions; small amino- or carboxyl-terminal extensions, such as an amino-terminal methionine residue; a small linker peptide; or a small extension that facilitates purification by changing net charge or another function, such as a poly-histidine tract, an antigenic epitope, or a binding domain.

In the above context, the term “small” independently designates a number of up to 25 amino acid residues. In preferred embodiments, the term “small” independently designates up to 24, 23, 22, 21, or up to 20 amino acid residues. In additional preferred embodiments, the term “small” independently designates up to 19, 18, 17, 16, 15, 14, 13, 12, 11, or up to 10 amino acid residues. In further preferred embodiments, the term “small” independently designates up to 9, 8, 7, 6, 5, 4, 3, 2, or up to 1 amino acid residue. In alternative embodiments, the term “small” independently designates up to 40, 39, 38, 37, 36, 35, 34, 33, 32, 31, 30, 29, 28, 27, 26, or up to 25 amino acid residues.

The lipase of the invention has an amino acid sequence which differs by no more than 25, 24, 23, 22, 21, 20, 19, 18, 17, 16, 15, 14, 13, 12, or no more than 11 amino acids from amino acids 1-269 of SEQ ID NO: 1; or, it differs from amino acids 1-269 of SEQ ID NO: 1 by no more than 10, 9, 8, 7, 6, 5, 4, 3, 2, or by no more than 1 amino acid; in either case, preferably, with the exception of the double substitution R231T+R233N in SEQ ID NO: 1, as defined above. In alternative embodiments, the lipase of the invention has an amino acid sequence which differs by no more than 40, 39, 38, 37, 36, 35, 34, 33, 32, 31, 30, 29, 28, 27, or no more than 26 amino acids from amino acids 1-269 of SEQ ID NO: 1, preferably, with the exception of the double substitution R231T+R233N in SEQ ID NO: 1, as defined above.

Examples of conservative substitutions are within the group of basic amino acids (arginine, lysine and histidine), acidic amino acids (glutamic acid and aspartic acid), polar amino acids (serine, threonine, glutamine and asparagine), hydrophobic amino acids (leucine, isoleucine, valine and alanine), aromatic amino acids (phenylalanine, tryptophan and tyrosine), and small amino acids (glycine, alanine, proline, serine, threonine, cysteine and methionine).

In the alternative, examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions which do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, AlaNal, Ser/Gly, Tyr/Phe, Ala/Pro, Lys/Arg, Asp/Asn, Leu/lIle, LeuNal, Ala/Glu, and Asp/Gly.

An example of a variant lipase of the invention which comprises a conservative substitution (exchange of one polar amino acid for another polar amino acid) is variant Asn33Gln (N33Q) of amino acids 1-269 of SEQ ID NO: 1. This is a non-glycosylated variant which is as efficient as SEQ ID NO: 1 for the purposes of the present invention (see Example 5). The present invention also relates to this variant lipase as such, as well as to the correspondingly substituted variants of amino acids −5-269, −4-269, −3-269, and 2-269 of SEQ ID NO: 1.

In a preferred embodiment, each of the substitutions in the variant lipase of the invention is conservative.

Examples of variant lipases of the invention which comprise small N-terminal extensions are amino acids −5-269 (−5 to +269), −4-269 (−4 to +269), and −3-269 (−3 to +269) of SEQ ID NO: 1, viz. with the N-terminals of SPI.., PIR.., and IRR.., respectively (see Example 5).

The lipase of the invention may also be an allelic variant of the lipase having amino acids 1-269 of SEQ ID NO: 2, preferably with the double-substitution T231R+N233R in SEQ ID NO: 2 (defined as above for SEQ ID NO: 1, mutatis mutandis).

The term allelic variant denotes any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene. Examples of allelic variants of the lipase of the invention are lipases derived from different strains of Humicola lanuginosa.

The lipase of the invention may also be a fragment of the lipase having amino acids 1-269 of SEQ ID NO: 1, whereby the fragment still has lipase activity. The term fragment is defined herein as a polypeptide having one or more amino acids deleted from the amino and/or carboxyl terminus of SEQ ID NO: 1, preferably from the mature part thereof (amino acids 1-269 thereof). Preferably, a small number of amino acids has been deleted, small being defined as explained above. More preferably, a fragment contains at least 244, 245, 246, 247, 248, 249, or at least 250 amino acid residues. Most preferably, a fragment contains at least 251, 252, 253, 254, 255, 256, 257, 258, 259, 260, 261, 262, 263, 264, 265, 266, 267, or at least 268 amino acid residues. In an alternative embodiment, a fragment contains at least 239, 240, 241, 242, or at least 243 amino acid residues.

An example of a variant lipase of the invention which is a fragment of amino acids 1-269 of SEQ ID NO: 1 is the variant having the amino acid sequence of amino acids 2-269 (+2 to +269) of SEQ ID NO: 1, viz. with the N-terminus of VSQ (see Example 5).

The invention also relates to

(a) a lipase for use as a medicament, wherein the lipase has at least 99.4% identity to amino acids 1-269 of SEQ ID NO: 1;

(b) a lipase comprising amino acids 1-269 of SEQ ID NO: 1, or a variant thereof, for use as a medicament, wherein the variant differs from amino acids 1-269 of SEQ ID NO: 1 by no more than twenty-five amino acids, and wherein, as compared to amino acids 1-269 of SEQ ID NO: 1, the variant comprises:

(i) at least one conservative substitution and/or insertion of one or more amino acids; and/or
(ii) at least one small deletion; and/or
(iii) at least one small N- or C-terminal extension; and/or wherein the variant is:
(iv) an allelic variant of the lipase having amino acids 1-269 of SEQ ID NO: 2; and/or
(v) a fragment of the lipase having amino acids 1-269 of SEQ ID NO: 1;
optionally with the proviso that the variant is not amino acids 1-269 of SEQ ID NO: 2; as well as corresponding compositions, methods and uses according to the invention of such lipases of (a) and (b). The percentage of identity is determined as described below.

The lipases with the following amino acid sequences are preferred examples of lipases of the invention: (i) amino acids +1 to +269 of SEQ ID NO: 1, (ii) amino acids −5 to +269 of SEQ ID NO: 1, (iii) amino acids −4 to +269 of SEQ ID NO: 1; (iv) amino acids −3 to +269 of SEQ ID NO: 1; (v) amino acids −2 to +269 of SEQ ID NO: 1; (vi) amino acids −1 to +269 of SEQ ID NO: 1, (vii) amino acids +2 to +269 of SEQ ID NO: 1, as well as (viii) any mixture of two or more of the lipases of (i)-(vii). In a particular embodiment, the lipase for use according to the invention is selected from the lipases of (i), (ii), and any mixture of (i) and (ii). Preferred mixtures of (i) and (ii) comprise at least 5%, preferably at least 10%, 20%, 30%, 40%, 50%, 60%, 70%, 80%, 90%, or at least 95% of lipase (i), the percentages being determined by N-terminal sequencing using the Edman method, as described in Example 5. Other preferred mixtures are: (a) compositions comprising 35-75%, preferably 40-70%, more preferably 45-65% of lipase (ii); (b) compositions comprising 20-60%, preferably 25-55%, more preferably 30-50%, most preferably 35-47% of lipase (i); (c) compositions comprising up to 30%, preferably up to 25%, more preferably up to 20%, most preferably up to 16% of lipase (vii); and (d) any combination of (a), (b), and/or (c), such as a composition comprising 45-65% of lipase (ii), 35-47% of lipase (i), and up to 16% of lipase (vii).

The present invention also relates to the isolated lipases (ii)-(vii) described above, as well as to any of the above-mentioned lipase mixtures and lipase compositions, in particular for pharmaceutical use as defined herein.

In still further particular embodiments, the lipase of the invention is used in combination with an additional lipase. Examples of additional lipases are mammalian lipases, and microbial lipases. A preferred mammalian lipase is pancreas extract, e.g. from swine or ox, such as pancreatin. The pancreatin may be used in the form of an uncoated (raw) product, or in the form of a formulated product (enteric coated (to provide resistance against gastric acid), or non-functionally coated (coated, but not to provide resistance against gastric acid)). Pancreatin potentially comprises still further enzymatic active constituents like pancreatic protease and/or pancreatic amylase. The microbial lipase may be, e.g., based on or derived from a bacterial or fungal lipase. Bacterial lipases can be derived from, e.g., Bacillus or Pseudomonas, fungal lipases can be derived from, e.g., strains of Rhizopus, Candida, or Humicola, such as Rhizopus delemar, Rhizopus javanicus, Rhizopus oryzae, or Humicola lanuginosa, in particular either of the products Lipase D2™ or Lipase D Amano 2000™ (lipase, EC 3.1.1.3) which are commercially available from Amano Pharmaceuticals, Japan.

The lipase of the invention may be used in combination with a protease, with or without an amylase as described below. The term “protease” is defined herein as an enzyme that hydrolyses peptide bonds. It includes any enzyme belonging to the EC 3.4 enzyme group (including each of the thirteen subclasses thereof, these enzymes being in the following referred to as “belonging to the EC 3.4.-.- group”).

Examples of proteases are mammalian proteases, and microbial proteases. A preferred mammalian protease is pancreas extract, e.g. from swine or ox, such as pancreatin. The pancreatin may be used in the form of an uncoated (raw) product, or in the form of a formulated product (enteric coated, or non-functionally coated). Pancreatin potentially comprises still further enzymatic active constituents like pancreatic lipase, BSSL (Bile Salt Stimulated Lipase), and/or pancreatic amylase.

The microbial protease may be, e.g., based on or derived from bacterial or fungal strains. The protease may in particular be derived from a strain of Aspergillus, such as Aspergillus oryzae or Aspergillus melleus, in particular the product Prozyme 6™ (neutral, alkaline protease EC 3.4.21.63) which is commercially available from Amano Pharmaceuticals, Japan. Examples of bacterial proteases are proteases from Bacillus and Nocardiopsis, such as the Bacillus licheniformis protease having the amino acid sequence of amino acids 1-274 of SEQ ID NO: 3, the Nocardiopsis sp. protease having the amino acid sequence of amino acids 1-188 of SEQ ID NO: 4, or the Nocardiopsis dassonviellei subsp. dassonvillei protease having the amino acid sequence of amino acids 1-188 of SEQ ID NO: 5. The protease of amino acids 1-274 of SEQ ID NO: 3 may, e.g., be prepared as described in DK patent application no. 2005 00930 entitled “Proteases for Pharmaceutical Use” and filed on Jun. 24, 2005 by Solvay Pharmaceuticals GmbH and Novozymes A/S. The proteases of amino acids 1-188 of SEQ ID NO: 4-5 may, e.g., be prepared as described in WO 2001/58276, or in WO 2004/111224.

In a preferred embodiment, the protease of the invention is at least 70% identical to a protease having, or comprising, either of (i) amino acids 1-274 of SEQ ID NO: 3, (ii) amino acids 1-188 of SEQ ID NO: 4, and/or (iii) amino acids 1-188 of SEQ ID NO: 5. In additional preferred embodiments of either of (i), (ii) or (iii), the degrees of identity is at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%. In alternative embodiments of either of (i), (ii), or (iii), the degrees of identity is at least about 50%, 51%, 52%, 53%, 54%, 55%, 56%, 57%, 58%, 59%, 60%, 61%, 62%, 63%, 64%, 65%, 66%, 67%, 68%, or at least 69%.

The lipase of the invention, with or without a protease as described above, may also be used in combination with an amylase.

In the present context, an amylase is an enzyme that catalyzes the endo-hydrolysis of starch and other linear and branched oligo- and polysaccharides. The amylose part of starch is rich in 1,4-alpha-glucosidic linkages, while the amylopectin part is more branched containing not only 1,4-alpha- but also 1,6-alpha-glucosidic linkages. In a particular embodiment, the amylase is an enzyme belonging to the EC 3.2.1.1 group.

In particular embodiments, the amylase is a mammalian amylase or a microbial amylase. An example of a mammalian amylase is pancreas extract, e.g. from swine or ox, such as pancreatin. The pancreatin may be used in the form of an uncoated (raw) product, or in the form of a formulated product (enteric coated, or non-functionally coated). Pancreatin potentially comprises still further enzymatic active constituents like pancreatic protease and/or pancreatic lipase. The microbial amylase may be, e.g., based on or derived from bacterial or fungal strains, such as Bacillus, Pseudomonas, Aspergillus, or Rhizopus.

The amylase may in particular be derived from a strain of Aspergillus, such as Aspergillus niger, Aspergillus oryzae or Aspergillus melleus, for example either of the products Amylase A1™ derived from Aspergillus oryzae which is commercially available from Amano Pharmaceuticals, Japan, or Amylase EC™ derived from Aspergillus melleus which is commercially available from Extract-Chemie, Germany.

Preferred amylases are (i) an amylase comprising amino acids 1-481 of SEQ ID NO: 6 (such as amino acids 1-481, 1-484, or 1-486 thereof), amino acids 1-481 of SEQ ID NO: 7, and/or amino acids 1-483 of SEQ ID NO: 8. In a preferred embodiment, the amylase is an amylase having, or comprising an amino acid sequence being, at least 70% identical to either of (i) amino acids 1-481 of SEQ ID NO: 6, (ii) amino acids 1-481 of SEQ ID NO: 7, and/or (iii) amino acids 1-483 of SEQ ID NO: 8. The amylases of SEQ ID NOs: 6-8 may, e.g., be prepared as described in co-pending DK application no. 2005 00931 entitled “Amylases for Pharmaceutical Use” and filed on Jun. 24, 2005 by Solvay Pharmaceuticals GmbH and Novozymes A/S. In additional preferred embodiments of either of (i), (ii), or (iii), the degrees of identity are at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%. In alternative embodiments of either of (i), (ii), or (iii), the degrees of identity are at least 50%, 51%, 52%, 53%, 54%, 55%, 56%, 57%, 58%, 59%, 60%, 61%, 62%, 63%, 64%, 65%, 66%, 67%, 68%, or at least 69%.

In one embodiment, the present invention relates to a lipase in combination with a protease and/or an amylase, wherein (i) the lipase comprises amino acids 2-269 of SEQ ID NO: 1; (ii) the protease is a protease selected from the group consisting of a) a protease having amino acids 1-274 of SEQ ID NO: 3, b) a protease having amino acids 1-188 of SEQ ID NO: 4, and c) a protease having amino acids 1-188 of SEQ ID NO: 5; (iii) the amylase is an amylase selected from the group consisting of a) an amylase comprising amino acids 1-481 of SEQ ID NO: 6, b) an amylase having amino acids 1-481 of SEQ ID NO: 7, and c) an amylase having amino acids 1-483 of SEQ ID NO: 8.

For the purposes of the present invention, particularly preferred combinations of enzymes are the following: (i) A lipase comprising amino acids 1-269, or 2-269, of SEQ ID NO: 1 in combination with a protease having amino acids 1-274 of SEQ ID NO: 3; (ii) a lipase comprising amino acids 1-269, or 2-269, of SEQ ID NO: 1 in combination with a protease having amino acids 1-188 of SEQ ID NO: 4; (iii) a lipase comprising amino acids 1-269, or 2-269, of SEQ ID NO: 1 in combination with a protease having amino acids 1-188 of SEQ ID NO: 5; (iv) a lipase comprising amino acids 1-269, or 2-269, of SEQ ID NO: 1 in combination with an amylase comprising amino acids 1-481 of SEQ ID NO: 6 (such as amino acids 1-481, 1-484, or 1-486 thereof); (v) a lipase comprising amino acids 1-269, or 2-269, of SEQ ID NO: 1 in combination with an amylase having amino acids 1-481 of SEQ ID NO: 7; (vi) a lipase comprising amino acids 1-269, or 2-269, of SEQ ID NO: 1 in combination with an amylase having amino acids 1-483 of SEQ ID NO: 8; (vii) a lipase comprising amino acids 1-269, or 2-269, of SEQ ID NO: 1 in combination with a protease having amino acids 1-274 of SEQ ID NO: 3 and an amylase comprising amino acids 1-481 of SEQ ID NO: 6 (such as amino acids 1-481, 1-484, or 1-486 thereof); (viii) a lipase comprising amino acids 1-269, or 2-269, of SEQ ID NO: 1 in combination with a protease having amino acids 1-274 of SEQ ID NO: 3 and an amylase having amino acids 1-481 of SEQ ID NO: 7; (ix) a lipase comprising amino acids 1-269, or 2-269, of SEQ ID NO: 1 in combination with a protease having amino acids 1-274 of SEQ ID NO: 3 and an amylase having amino acids 1-483 of SEQ ID NO: 8; (x) a lipase comprising amino acids 1-269, or 2-269, of SEQ ID NO: 1 in combination with a protease having amino acids 1-188 of SEQ ID NO: 4 and an amylase comprising amino acids 1-481 of SEQ ID NO: 6 (such as amino acids 1-481, 1-484, or 1-486 thereof); (xi) a lipase comprising amino acids 1-269, or 2-269, of SEQ ID NO: 1 in combination with a protease having amino acids 1-188 of SEQ ID NO: 4 and an amylase having amino acids 1-481 of SEQ ID NO: 7; (xii) a lipase comprising amino acids 1-269, or 2-269, of SEQ ID NO: 1 in combination with a protease having amino acids 1-188 of SEQ ID NO: 4 and an amylase having amino acids 1-483 of SEQ ID NO: 8; (xiii) a lipase comprising amino acids 1-269, or 2-269, of SEQ ID NO: 1 in combination with a protease having amino acids 1-188 of SEQ ID NO: 5 and an amylase comprising amino acids 1-481 of SEQ ID NO: 6 (such as amino acids 1-481, 1-484, or 1-486 thereof); (xiv) a lipase comprising amino acids 1-269, or 2-269, of SEQ ID NO: 1 in combination with a protease having amino acids 1-188 of SEQ ID NO: 5 and an amylase having amino acids 1-481 of SEQ ID NO: 7; and (xv) a lipase comprising amino acids 1-269, or 2-269, of SEQ ID NO: 1 in combination with a protease having amino acids 1-188 of SEQ ID NO: 5 and an amylase having amino acids 1-483 of SEQ ID NO: 8.

Other preferred combinations of enzymes are the following: (i) A lipase having at least 50% identity to amino acids 1-269 of SEQ ID NO: 1 in combination with a protease having at least 50% identity to amino acids 1-274 of SEQ ID NO: 3; (ii) a lipase having at least 50% identity to amino acids 1-269 of SEQ ID NO: 1 in combination with a protease having at least 50% identity to amino acids 1-188 of SEQ ID NO: 4; (iii) a lipase having at least 50% identity to amino acids 1-269 of SEQ ID NO: 1 in combination with a protease having at least 50% identity to amino acids 1-188 of SEQ ID NO: 5; (iv) a lipase having at least 50% identity to amino acids 1-269 of SEQ ID NO: 1 in combination with an amylase having at least 50% identity to amino acids 1-481 of SEQ ID NO: 6; (v) a lipase having at least 50% identity to amino acids 1-269 of SEQ ID NO: 1 in combination with an amylase having at least 50% identity to amino acids 1-481 of SEQ ID NO: 7; (vi) a lipase having at least 50% identity to amino acids 1-269 of SEQ ID NO: 1 in combination with an amylase having at least 50% identity to amino acids 1-483 of SEQ ID NO: 8; (vii) a lipase having at least 50% identity to amino acids 1-269 of SEQ ID NO: 1 in combination with a protease having at least 50% identity to amino acids 1-274 of SEQ ID NO: 3 and an amylase having at least 50% identity to amino acids 1-481 of SEQ ID NO: 6; (viii) a lipase having at least 50% identity to amino acids 1-269 of SEQ ID NO: 1 in combination with a protease having at least 50% identity to amino acids 1-274 of SEQ ID NO: 3 and an amylase having at least 50% identity to amino acids 1-481 of SEQ ID NO: 7; (ix) a lipase having at least 50% identity to amino acids 1-269 of SEQ ID NO: 1 in combination with a protease having at least 50% identity to amino acids 1-274 of SEQ ID NO: 3 and an amylase having at least 50% identity to amino acids 1-483 of SEQ ID NO: 8; (x) a lipase having at least 50% identity to amino acids 1-269 of SEQ ID NO: 1 in combination with a protease having at least 50% identity to amino acids 1-188 of SEQ ID NO: 4 and an amylase having at least 50% identity to amino acids 1-481 of SEQ ID NO: 6; (xi) a lipase having at least 50% identity to amino acids 1-269 of SEQ ID NO: 1 in combination with a protease having at least 50% identity to amino acids 1-188 of SEQ ID NO: 4 and an amylase having at least 50% identity to amino acids 1-481 of SEQ ID NO: 7; (xii) a lipase having at least 50% identity to amino acids 1-269 of SEQ ID NO: 1 in combination with a protease having at least 50% identity to amino acids 1-188 of SEQ ID NO: 4 and an amylase having at least 50% identity to amino acids 1-483 of SEQ ID NO: 8; (xiii) a lipase having at least 50% identity to amino acids 1-269 of SEQ ID NO: 1 in combination with a protease having at least 50% identity to amino acids 1-188 of SEQ ID NO: 5 and an amylase having at least 50% identity to amino acids 1-481 of SEQ ID NO: 6; (xiv) a lipase having at least 50% identity to amino acids 1-269 of SEQ ID NO: 1 in combination with a protease having at least 50% identity to amino acids 1-188 of SEQ ID NO: 5 and an amylase having at least 50% identity to amino acids 1-481 of SEQ ID NO: 7; and (xv) a lipase having at least 50% identity to amino acids 1-269 of SEQ ID NO: 1 in combination with a protease having at least 50% identity to amino acids 1-188 of SEQ ID NO: 5 and an amylase having at least 50% identity to amino acids 1-483 of SEQ ID NO: 8. In preferred embodiments of (i)-(xv), each degree of identity is, independently, at least 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%.

In one embodiment, the present invention relates to a combination of enzymes of a lipase together with a protease and/or an amylase, wherein (i) the lipase comprises an amino acid sequence which has at least 90% identity to amino acids 1-269 of SEQ ID NO: 1, with the proviso that the lipase is not amino acids 1-269 of SEQ ID NO: 2; (ii) the protease has at least 70% identity to a protease selected from the group consisting of a) a protease having amino acids 1-274 of SEQ ID NO: 3, b) a protease having amino acids 1-188 of SEQ ID NO: 4, and c) a protease having amino acids 1-188 of SEQ ID NO: 5; and/or (iii) the amylase has at least 70% identity to an amylase selected from the group consisting of a) an amylase having amino acids 1-481 of SEQ ID NO: 6, b) an amylase having amino acids 1-481 of SEQ ID NO: 7, and c) an amylase having amino acids 1-483 of SEQ ID NO: 8. In this embodiment, the lipase is preferably a) a lipase comprising amino acids 1-269 of SEQ ID NO: 1, or b) a lipase being a variant of amino acids 1-269 of SEQ ID NO: 1, wherein the variant differs from amino acids 1-269 of SEQ ID NO: 1 by no more than twenty-five amino acids, and wherein: (i) the variant comprises at least one conservative substitution and/or insertion of one or more amino acids as compared to amino acids 1-269 of SEQ ID NO: 1; and/or (ii) the variant comprises at least one small deletion as compared to amino acids 1-269 of SEQ ID NO: 1; and/or (iii) the variant comprises at least one small N- or C-terminal extension as compared to amino acids 1-269 of SEQ ID NO: 1; and/or (iv) the variant is an allelic variant of the lipase having amino acids 1-269 of SEQ ID NO: 2; and/or (v) the variant is a fragment of the lipase having amino acids 1-269 of SEQ ID NO: 1.

Generally, the lipase, protease, and amylase enzymes (hereinafter “the enzyme(s),” viz. the enzymes of the invention) may be natural or wild-type enzymes (obtained from animals, in particular mammals, for example human or swine enzymes; from plants, or from microorganisms), but also any mutants, variants, fragments etc. thereof exhibiting the desired enzyme activity, as well as synthetic enzymes, such as shuffled, hybrid, or chimeric enzymes, and consensus enzymes.

In a specific embodiment, the enzyme(s) are low-allergenic variants, designed to invoke a reduced immunological response when exposed to animals, including man. The term immunological response is to be understood as any reaction by the immune system of an animal exposed to the enzyme(s). One type of immunological response is an allergic response leading to increased levels of IgE in the exposed animal. Low-allergenic variants may be prepared using techniques known in the art. For example the enzyme(s) may be conjugated with polymer moieties shielding portions or epitopes of the enzyme(s) involved in an immunological response. Conjugation with polymers may involve in vitro chemical coupling of polymer to the enzyme(s), e.g. as described in WO 96/17929, WO 98/30682, WO 98/35026, and/or WO 99/00489. Conjugation may in addition or alternatively thereto involve in vivo coupling of polymers to the enzyme(s). Such conjugation may be achieved by genetic engineering of the nucleotide sequence encoding the enzyme(s), inserting consensus sequences encoding additional glycosylation sites in the enzyme(s) and expressing the enzyme(s) in a host capable of glycosylating the enzyme(s), see e.g. WO 00/26354. Another way of providing low-allergenic variants is genetic engineering of the nucleotide sequence encoding the enzyme(s) so as to cause the enzymes to self-oligomerize, effecting that enzyme monomers may shield the epitopes of other enzyme monomers and thereby lowering the antigenicity of the oligomers. Such products and their preparation is described e.g. in WO 96/16177. Epitopes involved in an immunological response may be identified by various methods such as the phage display method described in WO 00/26230 and WO 01/83559, or the random approach described in EP 561907. Once an epitope has been identified, its amino acid sequence may be altered to produce altered immunological properties of the enzyme(s) by known gene manipulation techniques such as site directed mutagenesis (see e.g. WO 00/26230, WO 00/26354 and/or WO 00/22103) and/or conjugation of a polymer may be done in sufficient proximity to the epitope for the polymer to shield the epitope.

In particular embodiments, the enzyme(s) are (i) stable at pH 2-8, preferably also at pH 3-7, more preferably at pH 4-6; (ii) active at pH 4-9, preferably 4-8; (iii) stable against degradation by pepsin and other digestive proteases (such as pancreas proteases, i.e., mainly trypsin and chymotrypsin); and/or (iv) stable and/or active in the presence of bile salts.

The lipase of the invention is preferably stable in the presence of bile salts, for example in the presence of 0.1-50 mM bile salts, preferably in the presence of 0.5-20 mM bile salts and even more preferred in the presence of 1-10 mM bile salts. The stability of the lipase in the presence of bile salts can for example be measured as remaining lipase activity after incubation in the presence of bile salts. A suitable method for measuring lipase stability in the presence of bile salts is given in the Example Section (measured for 60 minutes at pH 6.5 and 25° C. in the presence of 1.8 mM bile salts). Preferably, the remaining lipase activity of a lipase of the invention is at least a factor 1.1, 1.2, 1.4, 1.6, 1.8, 2.0, 2.2, 2.4, 2.6 or at least 2.7 higher than the corresponding remaining activity of a comparative lipase having the amino acid sequence of SEQ ID NO: 2, whereby the assay is preferably performed by incubation for 60 minutes at pH 6.5 and 25° C. in the presence of 1.8 mM bile salts.

The lipase of the invention is furthermore preferably stable in the presence of digestive proteases, in particular pepsin, more in particular at pH 3.0. A suitable method for measuring lipase stability at pH 3.0 and in the presence of porcine pepsin is given in the Example Section (measured for 3 hours at pH 3.0 and ambient temperature in the presence of 75 μg/mL porcine pepsin). Preferably, the residual lipase activity of a lipase of the invention is at least a factor 1.5, 2.0, 2.5, 3.0, 3.5, 4.0, or at least 4.5 higher than the corresponding residual activity of a comparative lipase having the amino acid sequence of SEQ ID NO: 2.

The term “in combination with” refers to the combined use according to the invention of the lipase, protease and/or amylase. The combined use can be simultaneous, overlapping, or sequential, these three terms being generally interpreted in the light of the prescription made by the physician.

The term “simultaneous” refers to circumstances under which the enzymes are active at the same time, for example when they are administered at the same time as one or more separate pharmaceutical products, or if they are administered in one and the same pharmaceutical composition.

The term “sequential” refers to such instances where one and/or two of the enzymes are acting first, and the second and/or third enzyme subsequently. A sequential action can be obtained by administering the enzymes in question as separate pharmaceutical formulations with desired intervals, or as one pharmaceutical composition in which the enzymes in question are differently formulated (compartmentalized), for example with a view to obtaining a different release time, providing an improved product stability, or to optimizing the enzyme dosage.

The term “overlapping” refers to such instances where the enzyme activity periods are neither completely simultaneous nor completely sequential, viz. there is a certain period in which the enzymes are both, or all, active.

The term “a”, for example when used in the context of the protease, lipase, and/or amylase of the invention, means at least one. In particular embodiments, “a” means “one or more,” or “at least one”, which again means one, two, three, four, five etc.

The relatedness between two amino acid sequences is described by the parameter “identity”.

For purposes of the present invention, the alignment of two amino acid sequences is determined by using the Needle program from the EMBOSS package (http://emboss.org) version 2.8.0. The Needle program implements the global alignment algorithm described in Needleman, S. B. and Wunsch, C. D. (1970) J. Mol. Biol. 48, 443-453. The substitution matrix used is BLOSUM62, gap opening penalty is 10, and gap extension penalty is 0.5.

The degree of identity between an amino acid sequence of the present invention (“invention sequence”; e.g. amino acids 1-269 of SEQ ID NO: 1) and a different amino acid sequence (“foreign sequence”; e.g. amino acids 1-269 of SEQ ID NO: 2) is calculated as the number of exact matches in an alignment of the two sequences, divided by the length of the “invention sequence” or the length of the “foreign sequence”, whichever is the shortest. The result is expressed in percent identity.

An exact match occurs when the “invention sequence” and the “foreign sequence” have identical amino acid residues in the same positions of the overlap (in the alignment example below this is represented by “|”). The length of a sequence is the number of amino acid residues in the sequence (e.g. the length of SEQ ID NO: 1 is 269).

In the, purely hypothetical, alignment example below, the overlap is the amino acid sequence “HTWGER-NL” of Sequence 1; or the amino acid sequence “HGWGEDANL” of Sequence 2. In the example a gap is indicated by a

Hypothetical alignment example:

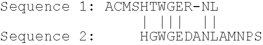

Accordingly, the percentage of identity of Sequence 1 to Sequence 2 is 6/12=0.5, corresponding to 50%.

In a particular embodiment, the percentage of identity of an amino acid sequence of a polypeptide with, or to, amino acids 1-269 of SEQ ID NO: 1 is determined by i) aligning the two amino acid sequences using the Needle program, with the BLOSUM62 substitution matrix, a gap opening penalty of 10, and a gap extension penalty of 0.5; ii) counting the number of exact matches in the alignment; iii) dividing the number of exact matches by the length of the shortest of the two amino acid sequences, and iv) converting the result of the division of iii) into percentage. The percentage of identity to, or with, other sequences of the invention such as amino acids 1-188 of SEQ ID NO: 4 is calculated in an analogous way.

In the alternative, the degree of identity between two amino acid sequences may be determined by the program “align” which is a Needleman-Wunsch alignment (i.e. a global alignment). The sequences are aligned by the program, using the default scoring matrix BLOSUM50. The penalty for the first residue of a gap is 12, and for further residues of a gap the penalties are 2. The Needleman-Wunsch algorithm is described in Needleman, S. B. and Wunsch, C. D., (1970), Journal of Molecular Biology, 48: 443-453, and the align program by Myers and W. Miller in “Optimal Alignments in Linear Space” CABIOS (computer applications in the biosciences) (1988) 4:11-17. “Align” is part of the FASTA package version v20u6 (see W. R. Pearson and D. J. Lipman (1988), “Improved Tools for Biological Sequence Analysis”, PNAS 85:2444-2448, and W. R. Pearson (1990) “Rapid and Sensitive Sequence Comparison with FASTP and FASTA,” Methods in Enzymology 183:63-98).

The degree of identity between a sample, or test, sequence of any of the enzyme(s) of the invention and a specified sequence may be determined as follows: The two sequences are aligned using the program “align.” The number of perfect matches (“N-perfect-match”) in the alignment is determined (a perfect match means same amino acid residue in same position of the alignment). The common length of the two aligned sequences is also determined, viz. the total number of amino acids in the alignment (the overlap), including trailing and leading gaps created by the alignment, if any (“N-overlap”). The degree of identity is calculated as the ratio between “N-perfect-match” and “N-overlap” (for conversion to percentage identity, multiply by 100).

The degree of identity between the sample, or test, sequence and a specified sequence may also be determined as follows: The sequences are aligned using the program “align.” The number of perfect matches (“N-perfect-match”) in the alignment is determined (a perfect match means same amino acid residue in same position of the alignment). The length of the sample sequence (the number of amino acid residues) is determined (“N-sample”). The degree of identity is calculated as the ratio between “N-perfect-match” and “N-sample” (for conversion to percentage identity, multiply by 100).

The degree of identity between the sample, or test, sequence and a specified sequence may also be determined as follows: The sequences are aligned using the program “align.” The number of perfect matches (“N-perfect-match”) in the alignment is determined (a perfect match means same amino acid residue in same position of the alignment). The length of the specified sequence (the number of amino acid residues) is determined (“N-specified”). The degree of identity is calculated as the ratio between “N-perfect-match” and “N-specified” (for conversion to percentage identity, multiply by 100).

Preferably, the overlap is at least 20% of the specified sequence (“N-overlap” as defined above, divided by the number of the amino acids in the specified sequence (“N-specified”), and multiplied by 100), more preferably at least 25%, 30%, 35%, 40%, 45%, 50%, 55%, 60%, 65%, 70%, 75%, 80%, 85%, 90%, or at least 95%. This means that at least 20% (preferably 25-95%) of the amino acids of the specified sequence end up being included in the overlap, when the sample sequence is aligned to the specified sequence.

In the alternative, the overlap is at least 20% of the specified sequence (“N-overlap” as defined above, divided by “N-sample” as defined above, and multiplied by 100), more preferably at least 25%, 30%, 35%, 40%, 45%, 50%, 55%, 60%, 65%, 70%, 75%, 80%, 85%, 90%, or at least 95%. This means that at least 20% (preferably 25-95%) of the amino acids of the sample sequence end up being included in the overlap, when aligned against the specified sequence.

The activity of the enzyme(s) of the invention can be measured using any suitable assay. Generally, assay-pH and assay-temperature may be adapted to the enzyme in question. Examples of assay-pH-values are pH 2, 3, 4, 5, 6, 7, 8, 9, 10, 11, or 12. Examples of assay-temperatures are 30, 35, 37, 40, 45, 50, 55, 60, 65, 70, 80, 90, or 95° C. Preferred pH values and temperatures are in the physiological range, such as pH values of 4, 5, 6, 7, or 8, and temperatures of 30, 35, 37, or 40° C.

Examples of suitable enzyme assays are included in the experimental part. Other examples are the FIP or Ph.Eur. assays for protease and amylase activity. These assays are, e.g., described in co-pending applications DK 2005 00930 and DK 2005 00931, respectively.

### Medicament

In the present context, the term “medicament” means a compound, or mixture of compounds, that treats, prevents and/or alleviates the symptoms of disease, preferably treats and/or alleviates the symptoms of disease. The medicament may be prescribed by a physician, or it may be an over-the-counter product.

### Pharmaceutical Compositions

Isolation, purification, and concentration of the enzyme(s) of the invention may be carried out by conventional means. For example, they may be recovered from a fermentation broth by conventional procedures including, but not limited to, centrifugation, filtration, extraction, spray-drying, evaporation, or precipitation, and further purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulphate precipitation), SDS-PAGE, or extraction (see, e.g., Protein Purification, J.-C. Janson and Lars Ryden, editors, VCH Publishers, New York, 1989).

For example, the lipase of SEQ ID NO: 1 may, e.g., be prepared on the basis of U.S. Pat. No. 5,869,438 (in which SEQ ID NO: 1 is a DNA sequence encoding the lipase of SEQ ID NO: 2 herein), viz. by recombinant expression in a suitable host cell of a DNA sequence which is a modification of SEQ ID NO: 1 of the US patent, the modification reflecting the amino acid differences between SEQ ID NO: 1 and 2 herein. Such modifications can be made by site-directed mutagenesis, as is known in the art.

In a particular embodiment, concentrated solid or liquid preparations of each of the enzyme(s) are prepared separately. These concentrates may also, at least in part, be separately formulated, as explained in more detail below.

In a further particular embodiment, the enzyme(s) are incorporated in the pharmaceutical compositions of the invention in the form of solid concentrates. The enzyme(s) can be brought into the solid state by various methods as is known in the art. For example, the solid state can be either crystalline, where the enzyme molecules are arranged in a highly ordered form, or a precipitate, where the enzyme molecules are arranged in a less ordered, or disordered, form.

Crystallization may, for example, be carried out at a pH close to the pI of the enzyme(s) and at low conductivity, for example 10 mS/cm or less, as described in EP 691982. In a particular embodiment, the lipase for use according to the invention is a crystalline lipase, which can be prepared as described in Example 1 of EP 600868 B1. The lipase crystals may furthermore be cross-linked as described in WO 2006/044529.

Various precipitation methods are known in the art, including precipitation with salts, such as ammonium sulphate, and/or sodium sulphate; with organic solvents, such as ethanol, and/or isopropanol; or with polymers, such as PEG (Poly Ethylene Glycol). In the alternative, the enzyme(s) can be precipitated from a solution by removing the solvent (typically water) by various methods known in the art, e.g. lyophilization, evaporation (for example at reduced pressure), and/or spray drying.

In a further particular embodiment, the solid concentrate of the enzyme(s) has a content of active enzyme protein of at least 50% (w/w) by reference to the total protein content of the solid concentrate. In still further particular embodiments, the content of active enzyme protein, relative to the total protein content of the solid concentrate is at least 55, 60, 65, 70, 75, 80, 85, 90, or at least 95% (w/w). The protein content can be measured as is known in the art, for example by densitometer scanning of coomassie-stained SDS-PAGE gels, e.g. using a GS-800 calibrated densitometer from BIO-RAD; by using a commercial kit, such as Protein Assay ESL, order no. 1767003, which is commercially available from Roche; or on the basis of the method described in Example 8 of WO 01/58276.

Preferably, the lipase enzyme protein constitutes at least 50%, more preferably at least 55, 60, 65, 70, 75, 80, 85, 90, 92, 94, 95, 96, or at least 97% of the protein spectrum of the solid lipase concentrate for use according to the invention, as measured by densitometer scanning of a coomassie-stained SDS-PAGE gel. For the lipase expressed in Aspergillus and comprising a mixture of the various N-terminal forms of SEQ ID NO: 1 as explained in Example 5, the relevant band on an SDS-PAGE gel is located corresponding to a molecular weight of 34-40 kDa. For the non-glycosylated variant of SEQ ID NO: 1, N33Q, the relevant band is located at around 30 kDa.

A pharmaceutical composition of the invention comprises the enzyme(s), preferably in the form of concentrated enzyme preparations, more preferably solid concentrates, together with at least one pharmaceutically acceptable auxiliary, or subsidiary, material such as (i) at least one carrier and/or excipient; or (ii) at least one carrier, excipient, diluent, and/or adjuvant. Non-limiting examples of, optional, other ingredients, all pharmaceutically acceptable, are disintegrators, lubricants, buffering agents, moisturizing agents, preservatives, flavouring agents, solvents, solubilizing agents, suspending agents, emulsifiers, stabilizers, propellants, and vehicles.

Generally, depending i.a. on the medical indication in question, the composition of the invention may be designed for all manners of administration known in the art, preferably including enteral administration (through the alimentary canal). Thus, the composition may be in solid, semi-solid, liquid, or gaseous form, such as tablets, capsules, powders, granules, microspheres, ointments, creams, foams, solutions, suppositories, injections, inhalants, gels, microspheres, lotions, and aerosols. The medical practitioner will know to select the most suitable route of administration and of course avoid potentially dangerous or otherwise disadvantageous administration routes.

The following methods and auxiliary materials are therefore also merely exemplary and are in no way limiting.

For solid oral preparations, the enzyme(s) can be used alone or in combination with appropriate additives to make pellets, micropellets, tablets, microtablets, powders, granules or capsules, for example, with conventional carriers, such as lactose, mannitol, corn starch, or potato starch; with excipients or binders, such as crystalline, or microcrystalline, cellulose, cellulose derivatives, acacia, corn starch, or gelatins; with disintegrators, such as corn starch, potato starch, or sodium carboxymethylcellulose; with lubricants, such as carnauba wax, white wax, shellac, waterless colloid silica, polyethylene glycol (PEGs, also known under the term macrogol) from 1500 to 20000, in particular PEG 4000, PEG 6000, PEG 8000, povidone, talc, monolein, or magnesium stearate; and if desired, with diluents, adjuvants, buffering agents, moistening agents, preservatives such as methylparahydroxybenzoate (E218), colouring agents such as titanium dioxide (E171), and flavouring agents such as saccharose, saccharin, orange oil, lemon oil, and vanillin. Oral preparations are examples of preferred preparations for treatment of the medical indication of PEI.

The enzyme(s) can also, quite generally, be formulated into liquid oral preparations, by dissolving, suspending, or emulsifying them in an aqueous solvent such as water, or in non-aqueous solvents such as vegetable or other similar oils, synthetic aliphatic acid glycerides, esters of higher aliphatic acids, propylene glycol, polyethylene glycol such as PEG 4000, or lower alcohols such as linear or ramified C1-C4 alcohols, for example 2-propanol; and if desired, with conventional subsidiary materials or additives such as solubilizers, adjuvants, diluents, isotonic agents, suspending agents, emulsifying agents, stabilizers, and preservatives.

Furthermore, the enzyme(s) can generally be made into suppositories for rectal administration by mixing with a variety of bases such as emulsifying bases or water-soluble bases. The suppository can include vehicles such as cocoa butter, carbowaxes and polyethylene glycols, which melt at body temperature, yet are solidified at room temperature.

The use of liposomes as a delivery vehicle is another method of possible general interest. The liposomes fuse with the cells of the target site and deliver the contents of the lumen intracellularly. The liposomes are maintained in contact with the cells for sufficient time for fusion, using various means to maintain contact, such as isolation, binding agents, and the like. In one aspect of the invention, liposomes are designed to be aerosolized for pulmonary administration. Liposomes may be prepared with purified proteins or peptides that mediate fusion of membranes, such as Sendai virus or influenza virus, etc. The lipids may be any useful combination of known liposome forming lipids, including cationic or zwitterionic lipids, such as phosphatidylcholine. The remaining lipid will normally be neutral or acidic lipids, such as cholesterol, phosphatidyl serine, phosphatidyl glycerol, and the like. For preparing the liposomes, the procedure described by Kato et al. (1991) J. Biol. Chem. 266:3361 may be used.

Unit dosage forms for oral or rectal administration such as syrups, elixirs, powders, and suspensions may be provided wherein each dosage unit, for example, teaspoonful, tablespoonful, capsule, tablet or suppository, contains a predetermined amount of the enzyme(s). Similarly, unit dosage forms for injection or intravenous administration may comprise the enzyme(s) in a composition as a solution in sterile water, normal saline, or another pharmaceutically acceptable carrier.

The term “unit dosage form”, as used herein, refers to physically discrete units suitable as unitary dosages for human and animal subjects, each unit containing a predetermined quantity of enzyme(s) in an amount sufficient to produce the desired effect.

In a particular embodiment, the pharmaceutical composition of the invention is for enteral, preferably oral, administration.

In further particular embodiments, the oral composition is (i) a liquid composition containing crystals of the enzyme(s); (ii) a liquid suspension of sediments of (highly) purified enzyme(s); (iii) a gel containing the enzyme(s) in solid or solubilized form; (iv) a liquid suspension of immobilized enzyme(s) or of enzymes adsorbed to particles and the like; or (v) a solid composition in the form of enzyme(s)-containing powder, pellets, granules, or microspheres, if desired in the form of tablets, capsules, or the like, that are optionally coated, for example with an acid-stable coating.

In another particular embodiment of the composition, the enzyme(s) are compartmentalized, viz. separated from each other, for example by means of separate coatings.

In a still further particular embodiment of the composition, the protease is separated from other enzyme components of the composition, such as the lipase, and/or the amylase.

The dosage of the enzyme(s) will vary widely, depending on the specific enzyme(s) to be administered, the frequency of administration, the manner of administration, the severity of the symptoms, and the susceptibility of the subject to side effects, and the like. Some of the specific enzymes may be more potent than others.

Examples of solid oral preparations of the enzyme(s) of the invention comprise: (i) a lipase of the invention having at least 90% identity to amino acids 1-269 of SEQ ID NO: 1; (ii) a protease having at least 70% identity to a protease selected from the group consisting of a) a protease having amino acids 1-274 of SEQ ID NO: 3, b) a protease having amino acids 1-188 of SEQ ID NO: 4, and c) a protease having amino acids 1-188 of SEQ ID NO: 5; and/or (iii) an amylase having at least 70% identity to an amylase selected from the group consisting of a) an amylase having amino acids 1-481 of SEQ ID NO: 6, b) an amylase having amino acids 1-481 of SEQ ID NO: 7, and c) an amylase having amino acids 1-483 of SEQ ID NO: 8; wherein preferably the anticipated daily clinical dosages of the enzymes of (i), (ii), and (iii) are as follows (all in mg enzyme protein per kg of bodyweight (bw)): For the lipase of (i): 0.01-1000, 0.05-500, 0.1-250, or 0.5-100 mg/kg bw; for the amylase of (ii): 0.001-250, 0.005-100, 0.01-50, or 0.05-10 mg/kg bw; for the protease of (iii): 0.005-500, 0.01-250, 0.05-100, or 0.1-50 mg/kg bw.

A preferred example of solid oral preparations of the enzyme(s) of the invention comprise: (i) a lipase comprising amino acids 2-269 of SEQ ID NO: 1, and (ii) an amylase comprising amino acids 1-481 of SEQ ID NO: 6, and/or (iii) a protease comprising, preferably having, amino acids 1-274 of SEQ ID NO: 3.

Examples of anticipated daily clinical dosages of the enzymes of (i), (ii), and (iii) are as follows (all in mg enzyme protein per kg of bodyweight (bw)): For the lipase of (i): 0.1-250, 0.5-100, or 1-50 mg/kg bw; for the amylase of (ii): 0.01-50, 0.05-10, or 0.1-5 mg/kg bw; for the protease of (iii): 0.05-100, 0.1-50, or 0.5-25 mg/kg bw.

The amide (peptide) bonds, as well as the amino and carboxy termini, may be modified for greater stability on oral administration. For example, the carboxy terminus may be amidated.

Particular embodiments of pharmaceutical compositions of the invention, suitable for the treatment of digestive disorders, PEI, pancreatitis, cystic fibrosis, diabetes type I, and/or diabetes type II, may be prepared by incorporating the enzyme(s) of the invention into pellets. The pellets may generally comprise from 10-90% (w/w, relative to the dry weight of the resulting pellets) of a physiologically acceptable organic polymer, from 10-90% (w/w, relative to the dry weight of the resulting pellets) of cellulose or a cellulose derivative, and from 80-20% (w/w, relative to the dry weight of the resulting pellets) of the enzyme(s), the total amount of organic polymer, cellulose or cellulose derivative and enzyme(s) making up to 100% in each case.

The physiologically acceptable organic polymer can be selected from the group consisting of polyethylene glycol 1500, polyethylene glycol 2000, polyethylene glycol 3000, polyethylene glycol 4000, polyethylene glycol 6000, polyethylene glycol 8000, polyethylene glycol 10000, polyethylene glycol 20000, hydroxypropyl methylcellulose, polyoxyethylene, copolymers of polyoxyethylene-polyoxypropylene and mixtures of said organic polymers. Polyethylene glycol 4000 is preferred as physiologically acceptable organic polymer.

The cellulose or a cellulose derivative can e.g. be selected from cellulose, cellulose acetate, cellulose fatty acid ester, cellulose nitrates, cellulose ether, carboxymethyl cellulose, ethyl cellulose, hydroxyethyl cellulose, hydroxypropyl cellulose, methyl cellulose, methyl ethylcellulose and methylhydroxypropyl cellulose. Cellulose, in particular microcrystalline cellulose is preferred as cellulose or cellulose derivative.

The resulting pellets may be coated with a suitable enteric coating, other non functional coating or be used directly without such coating. Further, the resulting pellets may be filled in capsules like hard gelatin capsules or gelatin free capsules of a suitable size for therapy of a disorder or disease as described in more detail above. In an embodiment of the invention, pellets produced from different enzyme types, in particular from lipase, protease and/or amylase may be filled into said capsules. While filling the capsules with the different enzyme types, the dosing of the single enzyme types (viz. lipase, protease or amylase) may be adapted to specific needs of a certain indication group or a certain patient subgroup by adding a specified amount of any of lipase, protease and/or amylase to the capsules, i.e. capsules may be produced which vary in their specific ratios of lipase:protease:amylase.

Preferred pharmaceutical compositions of the lipase of the invention are described in WO 2005/092370, in particular formulations comprising the preferred exhibients mentioned therein. In a particularly preferred embodiment, the pharmaceutical composition comprises a macrogolglyceride mixture of mono-, di- and tri-acylglycerides and polyethylene glycol (PEG) mono- and di-esters of aliphatic C6-C22 carboxylic acids, and also possibly small proportions of glycerol and free polyethylene glycol.

The polyethylene glycol (PEG) contained in the macrogolglyceride mixtures is preferably PEG which has on average 6 to at most 40 ethylene oxide units per molecule or a molecular weight of between 200 and 2000.

One further aspect of the invention provides for the pharmaceutical composition of the enzyme(s) of the invention to comprise a system consisting of surfactant, co-surfactant and lipophilic phase, the system having an HLB value (Hydrophilic-Lipophilic Balance) greater than or equal to 10 and a melting point greater than or equal to 30° C. In a preferred embodiment, the system has an HLB value of 10 to 16, preferably of 12 to 15, and has a melting point of between 30 and 600° C., preferably between 40 and 500° C. In particular, the system characterised by HLB value and melting point is a mixture of mono-, di- and triacylgylcerides and mono- and diesters of polyethylene glycol (PEG) with aliphatic carboxylic acids with 8 to 20, preferably 8 to 18, carbon atoms, whereby the polyethylene glycol preferably has about 6 to about 32 ethylene oxide units per molecule, and the system optionally contains free glycerin and/or free polyethylene glycol. The HLB value of such a system is preferably regulated by the chain length of the PEG. The melting point of such a system is regulated by the chain length of the fatty acids, the chain length of the PEG and the degree of saturation of the fatty-acid chains, and hence the starting oil for the preparation of the macrogolglyceride mixture.

“Aliphatic C8-C18 carboxylic acids” designates mixtures in which caprylic acid (C8), capric acid (C10), lauric acid (C12), myristic acid (C14), palmitic acid (C16) and stearic acid (C18) are contained in a significant and variable proportion, if these acids are saturated, and the corresponding unsaturated C8-C18 carboxylic acids. The proportions of these fatty acids may vary according to the starting oils.

Such a mixture of mono-, di- and triacylgylcerides and mono- and diesters of polyethylene glycol (PEG) with aliphatic carboxylic acids with 8 to 18 carbon atoms can for example be obtained by a reaction between a polyethylene glycol with a molecular weight of between 200 and 1500 and a starting oil, the starting oil consisting of a triglyceride mixture with fatty acids which are selected from the group containing caprylic acid, capric acid, lauric acid, myristic acid, palmitic acid, stearic acid, oleic acid and linolenic acid, individually or as a mixture. Optionally, the product of such a reaction may also contain small proportions of glycerin and free polyethylene glycol.

Such mixtures are commercially available for example under the trade name Gelucire®. One advantageous embodiment of the invention provides that, of the products known under the trade name Gelucire®, in particular “Gelucire® 50/13” and/or “Gelucire® 44/14” represent suitable mixtures for use in the pharmaceutical preparations according to the invention.

Gelucire® 50/13 is a mixture with mono-, di- and triacylglycerides and mono- and diesters of polyethylene glycol, with palmitic acid (C16) and stearic acid (C18) at 40% to 50% and 48% to 58%, respectively making up the major proportion of bound fatty acids. The proportion of caprylic acid (C8) and capric acid (C10) is less than 3% in each case, and the proportion of lauric acid (C12) and myristic acid (C14) in each case is less than 5%.

Gelucire® 44/14 is a mixture with mono-, di- and triacylgylcerides and mono- and diesters of polyethylene glycol, the respective proportions of palmitic acid (C16) being 4 to 25%, stearic acid (C18) 5 to 35%, caprylic acid (C8) less than 15%, capric acid (C10) less than 12%, lauric acid (C12) 30 to 50% and myristic acid (C14) 5 to 25%. Gelucire® 44/14 can for example be prepared by an alcoholysis/esterification reaction using palm kernel oil and polyethylene glycol 1500.

A preferred embodiment of the present invention provides for a pharmaceutical composition of the enzyme(s) of the invention which comprises a system containing a mixture of mono-, di- and triacyl-glycerides and polyethylene glycol mono- and diesters of aliphatic C8-C18 carboxylic acids and also possibly small proportions of glycerin and free polyethylene glycol, the system having a melting point between 40° C. and 55° C. and an HLB value in the range between 12 and 15. More preferred, the system has a melting point between 44° C. and 50° C. and an HLB value in the range from 13-14. Alternatively, the system has a melting point around 44° C. and an HLB value of 14, or the system has a melting point around 50° C. and an HLB value of 13.

### Methods of Treatment

The lipase for use according to the invention, optionally in combination with a protease, and/or an amylase (the enzyme(s) of the invention), is useful in the therapeutic, and/or prophylactic, treatment of various diseases or disorders in animals. The term “animal” includes all animals, and in particular human beings. Examples of animals are non-ruminants, and ruminants, such as sheep, goat, and cattle, e.g. beef cattle, and cow. In a particular embodiment, the animal is a non-ruminant animal. Non-ruminant animals include mono-gastric animals, e.g. horse, pig (including, but not limited to, piglets, growing pigs, and sows); poultry such as turkey, duck and chicken (including but not limited to broiler chicks, layers); young calves; pets such as cat, and dog; and fish (including but not limited to salmon, trout, tilapia, catfish and carps; and crustaceans (including but not limited to shrimps and prawns). In a particular embodiment the animal is a mammal, more in particular a human being.

For example, the enzyme(s) are useful in the treatment of digestive disorders like maldigestion or dyspepsia that are often caused by a deficient production and/or secretion into the gastrointestinal tract of digestive enzymes normally secreted from, i.a., the stomach, and the pancreas.

Further, the enzyme(s) are particularly useful in the treatment of PEI. PEI can be verified using, i.a., the Borgstrom test (JOP. J Pancreas (Online) 2002; 3(5):116-125), and it may be caused by diseases and conditions such as pancreatic cancer, pancreatic and/or gastric surgery, e.g. total or partial resection of the pancreas, gastrectomy, post gastrointestinal bypass surgery (e.g. Billroth II gastroenterostomy); chronic pancreatitis; Shwachman Diamond Syndrome; ductal obstruction of the pancreas or common bile duct (e.g. from neoplasm); and/or cystic fibrosis (an inherited disease in which a thick mucus blocks the ducts of the pancreas). The enzyme(s) may also be useful in the treatment of acute pancreatitis.

The effect of the enzyme(s) on digestive disorders can be measured as generally described in EP 0600868, in which Example 2 describes an in vitro digestibility test for measuring lipase stability under gastric conditions, and Example 3 an in vitro digestibility test for lipase activity in the presence of bile salts. Corresponding tests can be set up for the protease and amylase. Also WO 02/060474 discloses suitable tests, for example (1) an in vitro test for measuring lipid digestion in a swine test feed, and (2) an in vivo trial with pancreas insufficient swine in which the digestibility of fat, protein and starch is measured.

In a particular embodiment, the effect of the lipase of the invention is measured using the full in vivo digestibility trial of Example 2.

As another example, the enzyme(s) are useful in the treatment of Diabetes mellitus type I, and/or type II, in particular for adjuvant treatment in a diabetes therapy of digestive disorders usually accompanying this disease, with a view to diminishing late complications.

The effect on Diabetes mellitus of the enzyme(s) may be determined by one or more of the methods described in WO 00/54799, for example by controlling the level of glycosylated haemoglobin, the blood glucose level, hypoglycaemic attacks, the status of fat-soluble vitamins like vitamins A, D and E, the required daily dosage of insulin, the body-weight index, and hyper glycaemic periods.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties.

### EXAMPLES

### Example 1

### Enzyme Assays

Assays for lipase, protease and amylase activity of porcine pancreatin have been published by the FIP (Fédération Internationale Pharmaceutique) as well as the European Pharmacopoeia and the United States Pharmacopeia. 1 FIP-unit=1 Ph.Eur.-unit (European Pharmacopoeia). The assays are described in, e.g.: Fédération Internationale Pharmaceutique, Scientific Section: International Commission for the standardisation of pharmaceutical enzymes. a) “Pharmaceutical Enzymes,” Editors: R. Ruyssen and A. Lauwers, E. Story Scientia, Ghent, Belgium (1978), b) European Pharmacopoeia. See also Deemester et al in Lauwers A, Scharpé S (eds): Pharmaceutical Enzymes, New York, Marcel Dekker, 1997, p. 343-385. Appropriate enzyme standards can be procured from: International Commission on Pharmaceutical Enzymes, Centre for Standards, Harelbekestraat 72, B-9000 Ghent.

The lipase FIP assay as well as other suitable assays for lipase, protease and amylase is described below.

### Lipase FIP Assay

For measuring lipolytic activity of pancreatin the method published in the European Pharmacopoeia 5.1 was used. Unless otherwise stated, for determination of the lipolytic activity of microbial lipases the assay for Rhizopus oryzae lipase published by the FIP was used.

### Lipase pNP Assay

Substrate: para-Nitro-Phenyl (pNP) Valerate

### Assay pH: 7.7

Assay temperature: 40° C.
Reaction time: 25 min

The digested product with yellow colour has a characteristic absorbance at 405 nm. Its quantity is determined by spectrophotometry. One lipase unit is the amount of enzyme which releases 1 micromole titratable butyric acid per minute under the given assay conditions. A more detailed assay description, AF95/6-GB, is available on request from Novozymes A/S, Krogshoejvej 36, DK-2880 Bagsvaerd, Denmark.

### Lipase LU Assay

In this assay, the lipase-catalysed degradation of 0.16M tributyrin (glycerol tributyrate, Merck 1.01958.000) at pH 7.00 and 30° C. (+/−1° C.) is followed by pH-stat titration of released butyric acid with 0.025 M de-gassed, CO₂-free sodium hydroxide (Sodium hydroxide titrisol, Merck 9956). The consumption of the titrant is recorded as a function of time.

The substrate is emulsified with a 0.6% w/v Gum arabic emulsifier (20.0 g Gum Arabic, 89.5 g NaCl, 2.05 g KH₂PO₄, add water to 1.5 l, leave until completely dissolved, add 2700 ml glycerol, adjust pH to 4.5. 90 ml of tributyrin is mixed with 300 ml gum arabic emulsifier and 1410 ml demineralised water and homogenised for 3 minutes using e.g. a Silverson emulsifier L4RT at 7000 rpm and then adjusted to pH 4.75). Lipase-samples are diluted first in 0.1M glycin buffer pH 10.8, next in demineralized water, aiming at an activity level of 1.5-4.0 LU/ml. 15 ml of the emulsified substrate solution is poured into the titration vessel. 1.0 ml sample solution is added, and pH is maintained at 7.0 during the titration. The amount of titrant added per minute to maintain a constant pH is measured. The activity calculation is based on the mean slope of the linear range of the titration curve. A standard of known activity may be used as a level check.

1 LU (lipase unit) is the amount of enzyme which releases 1 micro mole titratable butyric acid per minute under the assay conditions given above. 1 kLU (kilo Lipase Unit)=1000 LU.

A more detailed assay description, EB-SM-0095.02, is available on request from Novozymes A/S, Krogshoejvej 36, DK-2880 Bagsvaerd, Denmark.

### Lipase pH Stat Assay

This assay is based on the lipase-catalysed release of fatty acids from an olive oil emulsion in the presence of 0.65 mM bile salts. The substrate is emulsified with gum arabic as emulsifier (175 g olive oil emulsified with 630 ml gum arabic solution (474.6 g gum arabic, 64 g calcium chloride in 4000 ml water) for 15 min in a blender; after cooling to room temperature, pH is adjusted to pH 6.8-7.0 using 4 M NaOH).

For the determination, 19 ml of the emulsion and 10 ml bile salts solution (492 mg bile salts are dissolved in water and filled up to 500 ml) are mixed in the reaction vessel and heated to 36.9° C. to 37.5° C. Reaction is started by addition of 1.0 ml of enzyme solution. The released acid is titrated automatically at pH 7.0 by addition of 0.1 M sodium hydroxide for a total of 5 min. The activity is calculated from the slope of the titration curve between the 1st and the 5th minute. For calibration, a standard is measured at three different levels of activity.

### Protease Suc-AAPF-pNA Assay

### Substrate: Suc-MPF-pNA (Sigma S-7388).

Assay buffer: 100 mM succinic acid, 100 mM HEPES (Sigma H-3375), 100 mM CHES (Sigma C-2885), 100 mM CABS (Sigma C-5580), 1 mM CaCl₂, 150 mM KCl, 0.01% Triton X-100 adjusted to pH 9.0 with HCl or NaOH.
Assay temperature: 25° C.

300 μl diluted protease sample was mixed with 1.5 ml of the assay buffer and the activity reaction was started by adding 1.5 ml pNA substrate (50 mg dissolved in 1.0 ml DMSO and further diluted 45× with 0.01% TritonX-100) and, after mixing, the increase in A₄₀₅ was monitored by a spectrophotometer as a measurement of the protease activity. The protease samples were diluted prior to the activity measurement in order to ensure that all activity measurements fell within the linear part of the dose-response curve for the assay.

### Protease AU Assay

Denatured haemoglobin (0.65% (w/w) in urea-containing 6.7 mM KH₂PO₄/NaOH buffer, pH 7.50) is degraded at 25° C. for 10 minutes by the protease and un-degraded haemoglobin is precipitated with trichloroacetic acid (TCA) and removed by filtration. The TCA-soluble haemoglobin degradation products in the filtrate are determined with Folin & Ciocalteu's phenol reagent (1 volume of Folin-Ciocalteu Phenol Reagent Merck 9001.0500 to 2 volumes of demineralised water), which gives a blue colour with several amino acids (being measured at 750 nm). The activity unit (AU) is measured and defined by reference to a standard. The denatured haemoglobin substrate may be prepared as follows: 1154 g urea (Harnstoff, Merck 8487) is dissolved in 1000 ml demineralised water, 240.3 g NaOH is added and then, slowly, 63.45 g haemoglobin (Merck 4300) is added, followed by 315.6 g KH₂PO₄, and demineralised water ad 3260 g. pH is adjusted to 7.63. More details and a suitable Alcalase standard are available on request from Novozymes A/S, Krogshoejvej 36, DK-2880 Bagsvaerd, Denmark (assay no. EB-SM-0349.01).

### Amylase

Substrate: Phadebas tablets (Pharmacia Diagnostics; cross-linked, insoluble, blue-coloured starch polymer, which is mixed with bovine serum albumin and a buffer substance, and manufactured into tablets)

### Assay Temperature: 37° C.

Assay pH: 4.3 (or 7.0, if desired)
Reaction time: 20 min

After suspension in water the starch is hydrolyzed by the alpha-amylase, giving soluble blue fragments. The absorbance of the resulting blue solution, measured at 620 nm, is a function of the alpha-amylase activity. One Fungal alpha-Amylase Unit (1 FAU) is the amount of enzyme which breaks down 5.26 g starch (Merck, Amylum solubile Erg. B. 6, Batch 9947275) per hour at the standard assay conditions. A more detailed assay description, APTSMYQI-3207, is available on request from Novozymes A/S, Krogshoejvej 36, DK-2880 Bagsvaerd, Denmark.

### Example 2

### In Vivo Digestibility Trial

The purified Humicola lanuginosa lipase variant having amino acids 1-269 of SEQ ID NO: 1 (together with a minor amount of a derivative thereof comprising amino acids-5-269 of SEQ ID NO: 1) was tested in a full digestibility study in female Göttingen minipigs (Ellegaard). The efficacy was compared to that of the Humicola lanuginosa lipase of SEQ ID NO: 2 (described in U.S. Pat. No. 5,614,189). Pancreatic Exocrine Insufficiency (PEI) was induced in the minipigs by ligation of the pancreatic duct, and they were also fitted with an ileo-caecal re-entrant cannula, all under halothane anaesthesia and at a weight of about 25 kg, as described in Tabeling et al., J. 1999, Studies on nutrient digestibilities (pre-caecal and total) in pancreatic duct-ligated pigs and the effects of enzyme substitution, J. Anim. Physiol. A. Anim. Nutr. 82: 251-263; and in Gregory et al., J. 1999. Growth and digestion in pancreatic duct ligated pigs, Effect of enzyme supplementation in “Biology of the Pancreas in Growing Animals” (SG Pierzynowski & R. Zabielski eds), Elsevier Science BV, Amsterdam, pp 381-393. A period of at least 4 weeks was allowed for recovery from surgery, before studies were commenced. Prior to study begin, the PEI status of each pig was confirmed via the stool chymotrypsin test (commercially available from Immundiagnostik AG, Wiesenstrasse 4, D-64625 Bensheim, Germany, with catalogue No. K 6990).

During the studies, the pigs were housed in pens on a 12:12 h light-dark cycle and allowed free access to water and fed two meals/day.

To assess lipase efficacy, the pigs were fed a 250 g test meal containing: 180 g double-milled diet, Altromin 902006 plus 70 g soya oil (Roth), mixed with 1 liter of water, and 0.625 g Cr₂O₃ (chromic oxide marker) and into which differing amounts of one or other of the two lipases were mixed immediately before feeding. The amount of each lipase administered is shown in brackets in Table 1, viz. the activities in FIP U lipase/meal (lipase FIP units, see Example 1). The test meal contained 16.3% protein, 28.9% starch and 32.9% fat, and included vitamins, minerals and trace elements as per the nutritional requirement for pigs. Each enzyme dosage was fed for at least 14 days: i.e the pigs were fed the high-fat diet plus each new enzyme dosage for 9 days after which all faeces were collected over the next 5 days, weighed and stored at −20° C.

The frozen faeces from each pig were freeze dried, weighed again and milled. Aliquots of each of the 5 day milled samples (according to the daily faecal production) were then pooled and mixed together; i.e. giving one pooled sample for each pig for each dose of enzymes. From each pooled sample the content of dry matter and crude fat were determined (Naumann & Bassler 1993; Die chemische Untersuchung von Futtermittein, 3. edition, VDLUFA-Verlag, Darmstadt (VDLUFA=Verband Deutscher Landwirtschaftlicher Untersuchungs- und For-schungsanstalten). Dry matter was estimated by weight after freeze-drying followed by 8 h incubation at 103° C.; crude fat was determined gravimetrically after boiling for 30 min in conc. HCl followed by a 6 h extraction with petrol ether; Cr₂O₃ was oxidized to chromate and chromium content calculated as described by Petry and Rapp in Zeitung für Tierphysiologie (1970), vol. 27, p. 181-189. (Petry & Rapp 1970; Z. Tierphysiol. 27; 181-189) via extinction at 365 nm (spectrophotometer).

Digestibility values (coefficient of fat absorption; CFA) were estimated by the marker method according to the formula:

$$C F A ( \% ) = 100 - [ \frac{\% {Cr}_{2} {O}_{3} in feed \cdot \% fat in faeces}{\% {Cr}_{2} {O}_{3} in faeces \cdot \% fat in feed} \cdot 100 ]$$

TABLE 1
Influence of enzyme supplementation on CFA (Coefficient of Fat Absorption)

| Enzyme | | | | |
|---|---|---|---|---|
| Supplement | 0 | Low | Medium | High |
| No supplement | 29.2 ± 7.6 | | | |
| Humicola | | 51.1 +/− 9.8 | 57.3 +/− 7.1 | 73.0 +/− 1.9 |
| lanuginosa | | (155400 FIP U) | (388400 FIP U) | (1165510 FIP U) |
| lipase variant | | | | |
| (SEQ ID NO: 1) | | | | |
| Humicola | | 31.2 +/− 10.2 | 38.8 +/− 8.0 | 43.2 +/− 3.5 |
| lanuginosa | | (112000 FIP U) | (280000 FIP U) | (840000 FIP U) |
| lipase (SEQ ID NO: 2) | | | | |

From the results in Table 1 it is apparent that the lipase of SEQ ID NO: 1 performs much better than the known lipase of SEQ ID NO: 1. In particular, it is more effective to increase the amount of fat absorption than the known lipase of SEQ ID NO: 2.

The lipases of the invention caused a very strong and dose-dependent improvement in fat digestibility, already showing a highly efficient improvement at the lower dose tested.

### Example 3

### Pharmaceutical Compositions

### (A) High-Strength Pellets

A liquid lipase concentrate was prepared comprising approximately 59% of the lipase having amino acids −5 to 269 of SEQ ID NO: 1, 36% of the lipase having amino acids 1-269 of SEQ ID NO: 1, and 5% of the lipase having amino acids 2-269 of SEQ ID NO: 1 (determined by N-terminal sequencing, and confirmed by ESIMS (Electro spray Ionisation Mass Spectrometry, as described in Example 5). The preparation was estimated to be approximately 92% pure on a protein basis as judged by SDS-PAGE, viz. the total amount of the three variants of SEQ ID NO: 1 constituted approximately 92% of the total amount of protein in the concentrate. The liquid concentrate was spray-dried. The measured lipase protein content of the spray-dried powder was 52.6%. 1145 g of the spray-dried lipase powder was dry pre-mixed together with microcrystalline cellulose (458 g) and polyethylene glycol 4000 (Macrogol™ 4000; 687 g) in a commercially available mixer. Isopropyl alcohol (460 g; 100%) was added and the resulting wet mass was continued to be thoroughly mixed at room temperature. The homogenized mass was then extruded in a commercially available extruder which was fitted with a piercing die having a hole diameter of 0.8 mm to form cylindrical pellets. The bead temperature was not exceeding 50° C. while extruding. The extrudate produced was rounded to spherical pellets with a commercially available spheronizer by adding the necessary amount of isopropyl alcohol 100% (87 g). The pellets were dried at a product temperature of approximately 40° C. in a commercially available vacuum dryer (from Voetsch). The product temperature did not exceed 45° C. The dried pellets were then separated by using a mechanical sieving machine with 0.7 and 1.4 mm screens. The sieve fractions of ≧0.7 mm and ≦1.4 mm were collected and filled in portions of 200 mg pellets each in capsules of size 2. The lipase concentration of the resulting dry pellets was approximately 26% (w/w).

### (B) Lower-Strength Pellets

Similar to the example provided above (A), pellets with a lower content of lipase as drug substance were produced using 450 g of the same spray dried lipase preparation, microcrystalline cellulose (1350 g), polyethylen glycol 4000 (450 g), isopropyl alcohol for moistening (750 g) and isopropyl alcohol for rounding (119.5 g). The lipase concentration of the resulting dry pellets was approximately 11% (w/w).

The resulting pellets from examples (A) and (B) were tested for lipolytic activity by applying the Lipase pH-stat assay described in Example 1. No loss in lipolytic activity was found in the pellets in each case relative to the starting powdery lipase material.

The resulting pellets from examples (A) and (B) were then tested for disintegration according to Pharm. Eur. 2.9.1. (Section “Disintegration of tablets and capsules”) (test solution: 0.1 M malonic acid, pH 6.0-500 mL, 37° C.).

The disintegration of the pellets from example (A) was completed within 4 min. and the activity found at 15-60 min was within 99-101% of the initial activity.

The disintegration of the pellets from example (B) was completed within 20 min. and the activity found at 15-60 min was within 101-99% of the initial activity.

The results show that it is possible to formulate the lipases of the invention as pellets without loss of lipase activity.

(C) Pellets Formed with Gelucire

The pellets were produced using the melt pelletizing process, which should be described here briefly: 262.5 g Gelucire® 44/14 (Gattefossé) and 262.5 g Gelucire® 50/13 (Gattefossé) were melted in a beaker in a heat chamber at a temperature of approx. 65° C. 975 g of spray-dried lipase powder as described above were provided in a dual-jacket mixer at 48° C. Thereafter, the molten Gelucire was added and the compounds were mixed using different speed levels and finally cooled (melt pelletisation).

### Example 4

### Activity in the Presence of Bile Salts

The same two purified lipases as were used in Example 2 (i.e. SEQ ID NO: 1 of the invention, and SEQ ID NO: 2 for comparison) were tested in vitro for activity in the presence of bile salts as follows:

Bile salts (Product no. B 3301 from Sigma-Aldrich) was dissolved into 0.1 M buffer (bistris-HCl buffer) at pH 6.5 to form a 2 mM solution. 28 mg olive oil and 18.8 mg para-nitrophenyl-palmitate (pNP-palmitate, or pNPP) (olive oil: pNPP molar ratio 2:1) as substrate were dissolved into 100 ml hexane and 200 micro liter of the resulting solution were pipetted into wells of a 96-well microtiterplate. The microtiterplate plate was left under hood to let hexane evaporate overnight at approximately 25° C., leaving olive oil and pNPP coated inside wells. The lipases were diluted to 0.01 mg/ml. 200 micro liter of the bilesalt solution and 20 micro liter of the lipase solutions referred to above were added/mixed into the lipid-coated microtiterplate and incubated for 60 minutes. Enzyme-free blanks were run as controls for subtraction.

A yellow colour developed as a result of the lipase-catalyzed liberation of para-nitrophenyl (pNP) from the substrate. The absorbancy at 405 nm (A405) was measured, being accordingly a measure of the lipase activity of the sample.

The results are shown in Table 2 below. The figures are calculated as the average of triplicate determinations and with subtraction of the enzyme-free blanks.

TABLE 2

| Enzyme | A₄₀₅ |
|---|---|
| Lipase of the invention (SEQ ID NO: 1) | 0.19 +/− 15% |
| Comparative lipase (SEQ ID NO: 2) | 0.07 +/− 22% |

The results of Table 2 show that the lipase of the invention is more stable in the presence of bile salts than the comparative lipase.

### Example 5

### Purification and Characterization

The lipase of SEQ ID NO: 1 was expressed in Aspergillus oryzae and purified from the fermentation broth as described in Examples 22 and 23 of U.S. Pat. No. 5,869,438. A number of batches of purified lipase were analysed by SDS-PAGE, and the lipase was identified as the main protein band at 34-40 kDa. By densitometer scanning of coomassie-stained SDS-PAGE gels this band was found to constitute 92-97% of the protein spectrum. The densitometer was a GS-800 calibrated densitometer from BIO-RAD.

However, the following slightly different N-terminal forms of SEQ ID NO: 1 were identified by N-terminal sequencing of this main protein band, below listed according to abundance. The amount of the various forms was determined by N-terminal sequencing by comparing the initial yields of the different forms in the first cycle of Edman degradation. The yields of the five N-terminal forms in the samples are also indicated:

| | | |
|---|---|---|
| #1 SPIRREVSQDLF . . . | (amino acids −5-269 of SEQ ID NO: 1) | 45-65% |
| #2 EVSQDLF . . . | (amino acids 1-269 of SEQ ID NO: 1) | 35-47% |
| #3 VSQDLF . . . | (amino acids 2-269 of SEQ ID NO: 1) | <1% to 16% |
| #4 PIRREVSQDLF . . . | (amino acids −4-269 of SEQ ID NO: 1) | <1% |
| #5 IRREVSQDLF . . . | (amino acids −3-269 of SEQ ID NO: 1). | <1% |

The two major forms #1 and #2 were found in all batches, form #3 in some batches but not all, and forms #4 and #5 in very low amounts in some batches (close to or below the detection limit).

It is believed that these variants have been formed as a result of cleavage by endogenous Aspergillus host proteases. For example, #2 might have been formed due to cleavage of #1 by KexB protease, #3 by cleavage with KexB and afterwards by aminopeptidase, and #4 and #5 by cleavage with aminopeptidase.

The quantification based on N-terminal sequencing was confirmed by ESIMS (Electro Spray Ionisation Mass Spectrometry), which showed matching mass intensities.

The difference between #1, #2, and #3 result in different theoretical pI values of 5.45, 5.11, and 5.23, respectively. Accordingly, these three forms were separated by IEF (Iso Electric Focusing), viz. on a pH 3-7 IEF gel. The bands were confirmed by N-terminal sequencing of blotted IEF gels. IEF is accordingly an easy and fast method for detection and quantification of forms #1, #2, and #3 of SEQ ID NO: 1.

Forms #1 and #2 of SEQ ID NO: 1 were found to have the same specific activity in LU/mg enzyme protein. For determining specific lipase activity, the lipase activity in LU/ml of the pure preparations was determined using the LU-assay of Example 1. The protein content of a particular lipase (mg enzyme protein/ml) was determined by amino acid analysis as described below, and the specific activity (LU/mg) calculated as Activity (LU/ml)/AAA (mg/ml).

Amino Acid Analysis (AAA)/(mg/ml): The peptide bonds of the lipase sample were subjected to acid hydrolysis, followed by separation and quantification of the released amino acids on a Biochrom 20 Plus Amino Acid Analyser, commercially available from Bie & Berntsen A/S, Sandbaekvej 5-7, DK-2610 Roedovre, Denmark, according to the manufacturer's instructions. The amount of each individual amino acid was determined by reaction with ninhydrin.

ESIMS data of the various lipase batches also clearly showed a complex glycosylation pattern corresponding to high mannose glycosylation with a number of mass peaks separated by a molecular weight corresponding to one hexose.

SEQ ID NO:1 includes one putative N-glycosylation site (NIT), N being residue number 33 of SEQ ID NO: 1. In fungal expression hosts N-acetylglucosamine residues will be linked to N-residues in a NIT-sequence as a result of post-translational modification, and a number of mannose monomers (from 5 to 21) will in turn be attached to the N-acetylglucosamine residues. This leads to a great variation in molecular weight of individual glycosylated molecules. By ESIMS the molecular weight ranges from approximately 30-34 kDa. The theoretical molecular weights of #1 and #2 without glycosylation are 30.2 kDa, and 29.6 kDa, respectively. This means that when expressed in a non-glycosylating host the main band on an SDS-PAGE gel will be narrower and corresponding to a molecular weight of around 30 kDa.

Variant N33Q (a conservative substitution) of SEQ ID NO: 1 will not be glycosylated even if expressed in fungal hosts. The non-glycosylated N33Q variant of SEQ ID NO: 1 showed similar efficacy as SEQ ID NO: 1 in an in vivo lipase screening test.

### Example 6

### Stability and Efficacy In Vivo in the Presence of Protease

The stability and efficacy of the Humicola lanuginosa lipase variant of SEQ ID NO: 1 in the presence of protease was tested as follows:

The purified lipase described in Example 2 was tested in an in vivo trial as generally described in Example 2, except that dosage was according to lipase units estimated in the pancreatic FIP assay. Digestibility values (coefficient of fat absorption; CFA) were estimated as also described in Example 2.

The lipase was tested alone, and in combination with protease, in various dosage combinations. The protease used was the Bacillus licheniformis protease of SEQ ID NO: 3. The protease activity was determined by using the pancreatic FIP assay (see reference in Example 1).

The results are shown in Table 3 below, given as average CFA (%) values and with indication of the standard deviation (sd).

TABLE 3

| | Lipase dosage | Protease dosage | | |
|---|---|---|---|---|
| | (Pancreatic FIP | (Pancreatic FIP Units | | |
| Treatment | Units per meal) | per meal) | CFA (%) | sd |
| Untreated PEI | 0 | 0 | 21.7 | 4.5 |
| (Control) | | | | |
| Lipase alone | 107200 | 0 | 59.2 | 4.7 |
| Lipase + | 107200 | 1200 | 55.6 | 6.7 |
| Protease | | | | |
| Lipase + | 107200 | 2400 | 58.7 | 5.1 |
| Protease | | | | |
| Lipase alone | 780892 | 0 | 75.6 | 4.7 |
| Lipase + | 780892 | 9000 | 81.4 | 4.0 |
| Protease | | | | |
| Lipase + | 780892 | 18000 | 76.0 | 3.2 |
| Protease | | | | |

For each of the two lipase dosages tested there was no significant difference between the results without and with protease, in the two different dosages. It can therefore be concluded that the protease had no adverse effect on the lipase in vivo.

### Example 7

### Stability in the Presence of Digestive Protease

The stability of the purified lipase of the invention in vitro, in the presence of one of the major digestive proteases and at a physiologically relevant pH, was measured as described below, in comparison to the known lipase of SEQ ID NO: 2.

The stability was determined as residual activity after treatment with porcine pepsin at pH 3.0.

Each lipase sample was treated with 75 μg/mL porcine pepsin, 2 mM calcium chloride, 0.01% Triton X-100 in 25 mM citrate buffer, pH 3.0 (final treatment conditions). One part of each diluted sample (diluent=10 mM NaCl, 0.01% Triton X-100) was added to one part treatment solution, and an untreated sample (control) was made by adding one part diluted sample to one part diluent. All treated and untreated samples were incubated for 3 hours at ambient temperature (20-25° C.), followed by an assay for residual activity.

The activity assay was made with 1 mM 4-nitrophenol Palmitate as substrate and 1.2% Triton X-100, 4 mM calcium chloride in 100 mM TRIS buffer, pH 8.0. The assay was performed such that for the treated sample, 10 parts substrate was added to 1 part treated sample and 1 part diluent (0.01% Triton X-100, 10 mM NaCl). For the untreated sample, 10 parts substrate was added to 1 part sample in diluent and 1 part pH 3.0 treatment solution. OD was read at 405 nm and is a measure of the lipase activity of the sample.

The resulting percentage of residual activity (% RA) was calculated as the assay result for the treated sample, relative to the assay result for the untreated sample. The results are shown in Table 4 below. C.V. indicates the coefficient of variation, and n the number of repetitions.

TABLE 4

| Enzyme | Residual Activity % | % C.V. | n |
|---|---|---|---|
| Lipase of the invention (SEQ | 9.7 | 40.0 | 13 |
| ID NO: 1) | | | |
| Comparative lipase (SEQ ID NO: 2) | 2.3 | 20.7 | 8 |

Table 4 shows that the lipase of the invention is more stable at pH 3.0 and in the presence of porcine pepsin as compared to the known lipase.

1-18. (canceled)

19. An isolated lipase for use as a medicament, wherein the lipase has at least 90% identity to amino acids 1-269 of SEQ ID NO: 1, provided that the lipase is not amino acids 1-269 of SEQ ID NO: 2.

20. The lipase of claim 19, wherein
(a) the lipase comprises amino acids 1-269 of SEQ ID NO: 1, or
(b) the lipase is a variant of amino acids 1-269 of SEQ ID NO: 1, wherein the variant differs from amino acids 1-269 of SEQ ID NO: 1 by no more than twenty-five amino acids, and wherein:
(i) the variant comprises at least one conservative substitution and/or insertion of one or more amino acids as compared to amino acids 1-269 of SEQ ID NO: 1; and/or
(ii) the variant comprises at least one small deletion as compared to amino acids 1-269 of SEQ ID NO: 1; and/or
(iii) the variant comprises at least one small N- or C-terminal extension as compared to amino acids 1-269 of SEQ ID NO: 1; and/or
(iv) the variant is an allelic variant of the lipase having amino acids 1-269 of SEQ ID NO: 2; and/or
(v) the variant is a fragment of the lipase having amino adds 1-269 of SEQ ID NO: 1.

21. The lipase of claim 19, which comprises amino acids 2-269 of SEQ ID NO: 1.

22. A pharmaceutical composition comprising a lipase of claim 20 and a pharmaceutically acceptable auxiliary material.

23. The composition of claim 22, further comprising a protease.

24. The composition of claim 23, wherein the protease has at least 70% identity to a protease selected from the group consisting of
(a) a protease having amino acids 1-274 of SEQ ID NO: 3,
(b) a protease having amino acids 1-188 of SEQ ID NO: 4, and
(c) a protease having amino acids 1-188 of SEQ ID NO: 5.

25. The composition of claim 22, further comprising an amylase.

26. The composition of claim 25, wherein the amylase has at least 70% identity to an amylase selected from the group consisting of
(a) an amylase having amino acids 1481 of SEQ ID NO: 6,
(b) an amylase having amino acids 1-481 of SEQ ID NO: 7, and
(c) an amylase having amino acids 1-483 of SEQ ID NO: 8.

27. The composition of claim 22, further comprising a protease and an amylase.

28. The composition of claim 27, wherein
(a) the protease has at least 70% identity to a protease selected from the group consisting of
(i) a protease having amino acids 1-274 of SEQ ID NO: 3,
(ii) a protease having amino acids 1-188 of SEQ ID NO: 4, and
(iii) a protease having amino acids 1-188 of SEQ ID NO: 5; and
(b) the amylase has at least 70% identity to an amylase selected from the group consisting of
(i) an amylase having amino acids 1-481 of SEQ ID NO: 6,
(ii) an amylase having amino acids 1-481 of SEQ ID NO: 7, and
(iii) an amylase having amino acids 1-483 of SEQ ID NO: 8.

29. A method for the treatment of digestive disorders, pancreatic exocrine insufficiency, pancreatitis, cystic fibrosis, diabetes type I, and/or diabetes type II, comprising administering a therapeutically effective amount of a lipase of claim 20.

30. The method of claim 29, further comprising administering a therapeutically effective amount of a protease or an amylase.

31. The method of claim 29, further comprising administering a therapeutically effective amount of a protease and an amylase.
